(19) United States
(12) Patent Application Publication
(10) Pub. No.: US 2024/0268981 A1
(43) Pub. Date: Aug. 15, 2024

(54) ORTHOSIS

(71) Applicant: OTTO BOCK HEALTHCARE PRODUCTS GMBH, Wien (AT)

(72) Inventor: Christian BREUER-REUSCH, Wien (AT)

(21) Appl. No.: 18/559,607

(22) PCT Filed: May 18, 2022
(86) PCT No.: PCT/EP2022/063447

§ 371 Date: Nov. 8, 2023

(30) Foreign Application Priority Data
May 19, 2021 (DE) 10 2021 112 989.3

(51) Int. Cl. A61F 5/01

(52) U.S. Cl. CPC A61F 5/0111; A61F 2005/0165; A61F 2005/0197

(57) ABSTRACT

The invention relates to an orthosis with a foot part (10) and a lower-leg part (20), which are connected to each other via a spring (30), wherein the spring (30), starting from a neutral position, provides a higher resistance during dorsal flexion than during plantar flexion.

The invention relates to an orthosis with a foot part and a lower-leg part, which are connected to each other via a spring. Such an orthosis is often used as a foot lift orthosis, which is fitted to users with peroneal paresis in order to prevent what is called drop foot or to reduce its effects.

If the ankle joint lacks stability and if the muscle strength is not sufficient, it may be useful and necessary to use an ankle-foot orthosis (AFO). The ankle-foot orthosis maintains the foot in a relationship to the shinbone and prevents unwanted dorsal flexion as well as plantar flexion when the foot is unloaded. During walking, the orthosis supports the patient by maintaining a dorsally flexed position at heel strike and enabling push-off in the terminal stance phase. Unintentional pronation and supination of the foot is also avoided. If such orthoses extend over the knee joint, they are referred to as knee-ankle-foot orthoses (KAFO).

DE 603 15 698 T2 relates to an ankle-foot orthosis with a structural frame which has a foot plate, an inner part and an ankle part and is split into two frontal support elements, which are arranged frontally to the shinbone both medially and laterally. The ankle-foot orthosis can be fitted to the patient by means of a fastening device in the form of a Velcro strap.

Another AFO is known from DE 10 2012 011 466 A1, in which a foot part is connected to a lower-leg part via a spring. The users of such an orthosis have the advantage that the deformation energy stored in the spring is returned at toe-off for initiation of the swing phase. However, this advantage is limited in the sense that the foot can barely be moved in the neutral position and in particular in the swing phase. Furthermore, the individual configuration of an AFO is difficult and cannot be corrected at a later stage.

Orthoses with a lower-leg part and a foot part, which are coupled to each other via a joint and can be pivoted relative to each other about a defined pivot axis, have a complex structure, are comparatively heavy and require a large installation space.

The object of the present invention is therefore to make available an orthosis that is light, takes up little space, has a low degree of complexity and permits energy recovery along with optimized stiffness.

According to the invention, this object is achieved by an orthosis having the features of the main claim. Advantageous embodiments and developments of the invention are disclosed in the subclaims, the description and the figures.

In the orthosis with a foot part and a lower-leg part, which are connected to each other via a spring, provision is made that the spring, starting from a neutral position, provides a higher resistance during dorsal flexion than during plantar flexion. Plantar flexion is the movement that a foot makes when the tip of the foot is moved downward; dorsal flexion is the movement that the foot makes when the tip of the foot is moved in the direction of the shinbone. The neutral position of the orthosis is the position that an unloaded orthosis adopts, in particular an orthosis that is in the swing phase, without forces being exerted on the foot part and the lower-leg part, which would cause dorsal flexion or plantar flexion of the foot part. The spring connects the foot part, on which the foot is placed preferably completely, or at least partially, to the lower-leg part, which is equipped with appropriate fastening devices for supporting the foot part on the lower-leg part. The fastening devices are in particular buckles, straps, clasps or other form-fitting elements or components that at least partially engage around the lower leg. The foot is placed onto the foot part; the lower-leg part is placed onto the lower leg and fastened there. Contact surfaces or contact regions for a part of the lower leg are in particular arranged or formed on the lower-leg part, for example clasp-like contact regions, which have a large contact surface. The contact surface can be provided with padding. The spring connecting the foot part, which can be a rigid or elastic component, to the lower-leg part permits pivoting of lower leg to foot and thus of lower-leg part to foot part. The spring does not provide a defined pivot axis, but permits dorsal flexion and plantar flexion by a deformation of the spring. Starting from a neutral position, the spring provides a higher resistance during dorsal flexion than during plantar flexion, which makes it possible to restrict the plantar flexion as little as possible, for example in order to permit improved positioning of the foot when sitting or even at heel strike. Furthermore, on account of such different bending stiffness, the knee joint is less pressed into flexion when the user is standing on an inclined surface. At the same time, during roll-over and toe-off at the end of the stance phase, there is a pronounced return of energy due to the spring effect. The resulting stretching effect on the knee joint due to the stability in the direction of dorsal flexion is also desired. In addition, the calf muscles in AFO or KAFO users are often impaired in terms of their function, as a result of which the spring plays an important role in supporting the foot during dorsal flexion.

In one development, the spring is coupled to a force transmission element that transmits forces only when moved in the direction of dorsal flexion. The force transmission element is designed, for example, as a tension rod, cable, belt or telescopic rod and has the effect that a movement in the direction of plantar flexion takes place without a force transmission by the force transmission element. If a dorsal flexion movement is performed, that is to say the spring is bent in the opposite direction, the force transmission element is, for example, subjected to a tensile force, such that a stiffening of the spring and an increased resistance to dorsal flexion are made available. The force transmission element is advantageously designed rigidly with respect to the transmission of tensile forces, while it is resilient or flexible with respect to compressive forces, which can be easily realized, for example, via tensionally rigid tensile means such as belts, cables, ropes or even a telescopic rod.

In one embodiment, the force transmission element is designed or mounted in an adjustable manner. In a flexible, non-elastic embodiment of the force transmission element, the latter can be secured in different positions on the spring, or the fastening location can be designed to be adjustable or displaceable. The adjustability can be achieved, for example, via a thread, a sliding bearing, a rotary bearing on an eccentric, or by fastening in discrete positions on different fastening elements. Depending on the position, there is, for example, a different idle travel during displacement or an increased pretensioning of the spring in the direction of dorsal flexion.

In one embodiment, the force transmission element is assigned an actuator, by which the location of the force transmission and/or the time of the force transmission can be changed. The actuator adjusts the position of the force application of the force transmission element on the spring and/or the foot part, such that a differently high resistance is made available depending on the position of the foot part to the lower-leg part or on the bending of the spring. Likewise, when the force application point of the force transmission element is shifted, the location of the force application and thus also the possible resistance to plantar flexion and dorsal flexion is set. The actuator allows the effective length of the force transmission element to be changed, i.e. shortened or increased. If the length is shortened, the flexion resistance increases, so that an improved or stronger toe-off is possible, for example, when walking. By contrast, in the case of a desired lower stiffness, for example when sitting, the resistance can be reduced by a lengthening of the force transmission element, provided that the latter is designed as a tension element. This allows the stiffness of the orthosis structure to be adaptively matched to the gait situation or the usage situation.

In one embodiment, the spring has a plurality of spring elements, whereby an individual adaptation to the respective user is easily possible. In addition, the design of the spring with a plurality of spring elements affords increased variability in the adjustment of the respective resistances against plantar flexion and dorsal flexion.

In one embodiment, a plurality of spring elements are spaced apart from one another in the neutral position; these spring elements bear on one another during dorsal flexion. The distance of the spring elements from one another can be minimal; it is essential that an interface between the spring elements is formed and that the spring elements can separate from one another at least in part. If dorsal flexion takes place, the spring elements bear against one another or are pressed onto one another more strongly, resulting in a coupling of the individual spring elements. By virtue of the interaction between the individual spring elements during dorsal flexion of the foot part, the resistance of the spring element increases. During plantar flexion, the individual spring elements separate from one another and at least in part lift away from one another, so that the individual spring elements together provide a lower resistance to deformation than in a state when they are bearing on one another. The more spring elements are arranged separately from and parallel to one another, the more noticeable is this behavior.

In one embodiment, the spring elements are arranged next to one another or one behind another, in such a way that they bear on one another in succession during dorsal flexion of the foot part. This can happen, for example, if the distances between the spring elements are not uniform but are instead designed increasing in increments. As a result, with increasing dorsal flexion, two spring elements first of all come to bear on each other, then three spring elements, and then the other spring elements. The spring elements are brought into engagement or made effective one after another depending on the achieved flexion angle.

In one embodiment, a spacer is arranged between the spring elements and can be used to establish when two spring elements come to bear against each other and interact. The spacers can be elastic or deformable, such that a damping effect is made available when the spring elements move relative to one another and deform the spacer or spacers. This makes it possible to influence the resistance behavior of the spring and of the spring elements. In addition, the perceived increase of the bending stiffness and the increased resistance to deformation of the spring becomes less abrupt than without spacers. With exchangeable spacers, it is possible to adapt to different patients and different resistances, depending on the requirements or the area of use. Thus, adjustments can also be made after the orthosis has been produced.

If the spacer is mounted movably on one of the spring elements, the setting and adjustment can be easily done by an orthopedic technician or the user of the orthosis, so that individual needs can be met easily and cost-effectively. The spacer can also be used to change the shape and/or position of the force transmission element, as a result of which, in the case of a belt or rope, the so-called slack or sag or the pretensioning can be adjusted. The spacer itself or the spacers can additionally have elastic properties which, in combination with the other spring elements, influence the resistance to deformation.

For the adjustment and/or displacement of the spacer or the spacers, at least one actuator is assigned to the spacer or spacers. The actuator is designed, for example, as a motor drive, piezoelement, magnetic drive, solenoid or the like and acts on the spacer or spacers directly or via a component.

In one embodiment, the spring elements are designed as leaf springs, in particular as leaf springs made of a fiber composite material. The individual leaf springs can be coupled to one another at their ends or at least at one of their ends; the region that couples the spring elements to one another can be a part of a spring element, or of all the spring elements, from which the other spring elements extend in a corresponding same direction.

In one embodiment of the invention, the spring elements are arranged parallel to one another and, in the neutral position, are shaped in an arch curving counter to the direction of walking. The parallel arrangement refers to the parallel connection; a geometric parallelism may be present, but it is not necessary. The arch is curved convexly counter to the direction of walking or toward the rear end of the foot part, whereby the individual spring elements separate from one another during plantar flexion, so that a comparatively low resistance is made available by the spring, since the individual spring elements, in a state when moved away from one another, can deform more easily than under a reverse load, in which the spring elements bear on one another and together generate an increased resistance to dorsal flexion.

The spring elements can have different stiffnesses in order to provide a stepped profile of a resistance increase when the spring elements become effective in succession or are combined with one another in succession.

In one embodiment, the spring has a base spring element, on which the foot part and the lower-leg part are arranged, in particular attached thereto or integrally formed. The spring can be designed as a separate component and, after it has been produced, can be connected to the foot part and the lower-leg part permanently or in such a way as to be repeatedly releasable from and fastenable thereto. Reduced assembly effort, reduced weight and reduced complexity are achieved when the spring is integrally formed together with the foot part and the lower-leg part in the context of the manufacturing process, and a one-piece configuration of the base spring element with the foot part and the lower-leg part is produced by original forming. Basically, it is also possible to design the spring with the plurality of spring elements in a single piece by means of a cohesively bonded connection; the lower-leg part and the foot part can also be integrally formed.

If a base spring element is connected to or designed in one piece with the foot part and the lower-leg part, there is a possibility of a further spring element being mounted exchangeably on the base spring element. By virtue of the exchangeability of the further spring element, it is possible to carry out an individual adjustment or also to easily carry out a repair.

Exemplary embodiments are explained in more detail below with reference to the appended figures. The same reference signs designate the same components. In the figures:

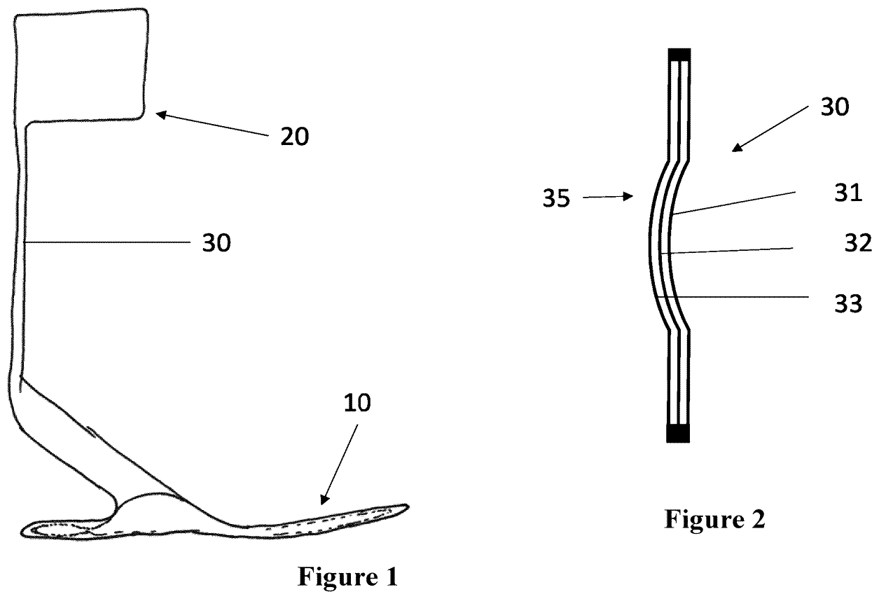

FIG. 1 shows a schematic representation of an AFO;

FIG. 2 shows an embodiment of a spring in isolation;

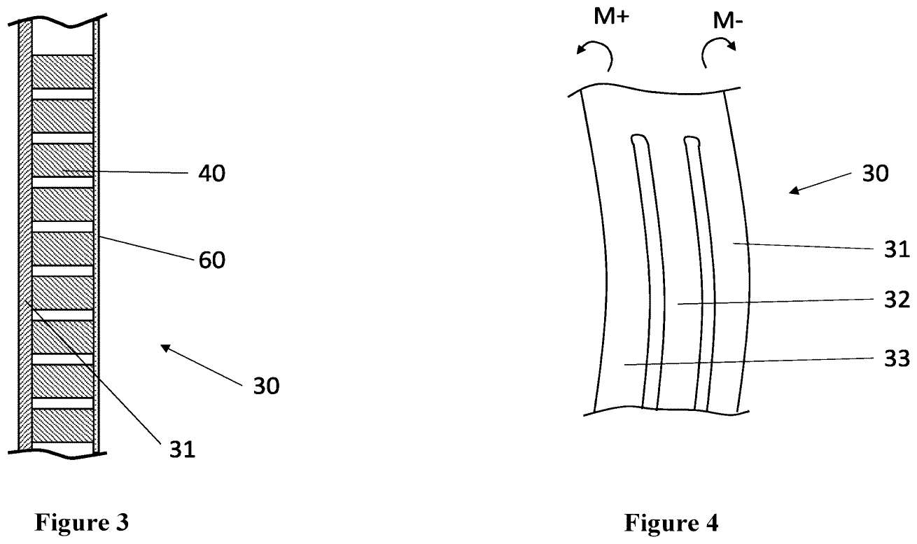

FIG. 3 shows a detailed view of a spring with a force transmission element;

FIG. 4 shows a detailed view of a spring with a plurality of spring elements;

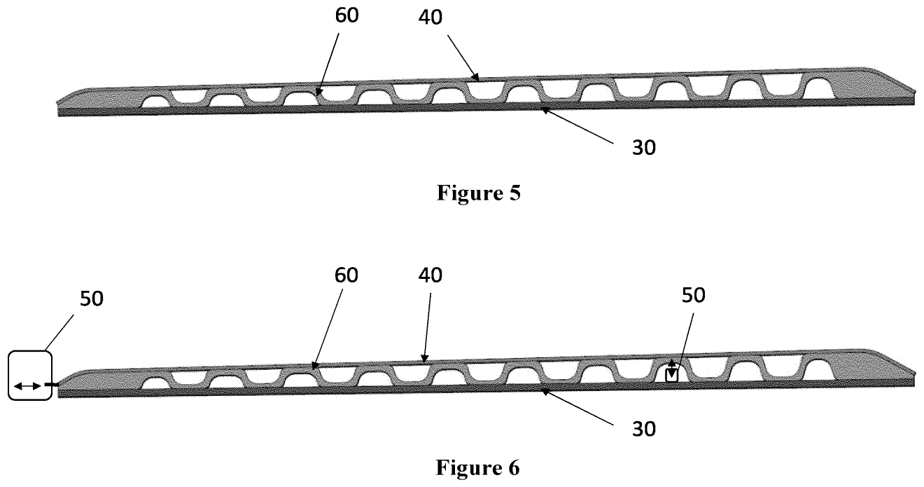

FIG. 5 shows a variant of the spring design; and

FIG. 6 shows a variant of FIG. 5 with actuators.

FIG. 1 is a schematic side view showing an orthosis in the form of an ankle-foot orthosis with a foot part 10 and with a lower-leg part 20 formed integrally on the latter. The foot part 10 has a flat contact surface on which the foot can be fully set down. The contact surface extends over the entire foot length in the illustrated embodiment. Alternatively, shorter contact surfaces can be provided. For example, the contact surface of the foot part 10 can end in the region of the ball of the foot before the toes or in the metatarsal region. From the center of the plate-shaped support surface, in the illustrated example from the medial side, a connecting strut extends obliquely to the rear and upward and merges there into a spring 30, which constitutes part of the lower-leg part 20. In an alternative design, two connecting struts are provided which, in the medial and lateral directions, extend obliquely upward to the rear; a lateral arrangement of a connecting strut is also an option. At the proximal end of the lower-leg part 20, a shell-like receptacle is formed for the calf. As an alternative to a rear arrangement of the shell on the calf muscle, it is possible to arrange a corresponding device or a corresponding receiving element in the region of the shinbone. By means of fastening devices not shown, such as belts, buckles or the like, the lower-leg part 20 is fixed to the lower leg (not shown).

The foot part 10 can also have a fastening device for securing a foot that is set down on the contact surface. In one embodiment, the heel region has a closed configuration, which achieves improved stability. As an alternative to the connection of the foot part 10 to the lower leg part 20 via the obliquely rearwardly extending strut, it is possible and provided to guide the lower-leg part 20 in the heel region of the foot plate upward in the proximal direction. In the exemplary embodiment shown, the foot part 10 and the lower-leg part 20 are formed in one piece from a fiber composite material. The spring 30 is thus an integral part of the lower-leg part 20 and forms the connection to the foot part 10. The spring 30 or the spring region permits a pivoting of the proximal end of the lower leg part 20 relative to the foot part 10 forward and rearward, the movement being effected by a deformation of the spring 30. A defined articulation axis is not formed by the spring 30.

As an alternative to a one-piece configuration of foot part 10, lower-leg part 20 and spring 30, these can also be designed as separate components. For example, the foot part 10 and the lower-leg part 20 can have receiving devices such as bores, recesses or plug-in sleeves, into which a respective end of the spring 30 is inserted and fixed. The fixing can, for example, be carried out permanently by means of adhesive bonding. Alternatively, the spring 30 can be mechanically secured and released via fastening elements such as bolts, screws, clip elements or the like. The spring and the foot part or the lower-leg part can form a one-piece component, which is then combined with the remaining component to form the orthosis.

FIG. 2 is a detailed representation of a spring 30 composed of a total of three spring elements 31, 32, 33, which are oriented substantially parallel to one another. All the spring elements 31, 32, 33 are arranged acting in parallel with one another and, in the exemplary embodiment shown, are interconnected at the upper, proximal end and the lower, distal end. The connection can be provided in a cohesively bonded manner by pouring or laminating or gluing. Alternatively, the individual spring elements 31, 32, 33 can be clamped or screwed together. All the spring elements 31, 32, 33 are designed as leaf springs and, in the exemplary embodiment shown, are made from a fiber composite material. By way of the spaced arrangement in the end regions, a clearance between the spring elements 31, 32, 33 is provided over the entire length. Alternatively, the separate spring elements 31, 32, 33 can bear against one another in the neutral position shown. It is also provided and possible that the distance between a first spring element 31 and a second spring element 32 is different than the distance between the second spring element 32 and a third spring element 33.

In the neutral position shown in FIG. 2, the spring 30 has an arch 35, which is convex in the direction of a rear region of the foot part 10. Such an arrangement has the effect that, in the event of a deformation of the end regions of the spring 30 during dorsal flexion, when the top of the foot part 10 moves toward the proximal, front end of the lower-leg part 20, the individual spring elements 31, 32, 33 are moved toward one another and the distance between the spring elements 31, 32, 33 decreases in particular in the region of the arch 35. During a reverse movement, the plantar flexion, the individual spring elements 31, 32, 33 are moved apart, so that the distance between the spring elements 31, 32, 33 is also increased, in particular in the region of the arch 35. The effect of this is that, on account of the separation of the spring elements 31, 32, 33 from one another, only a reduced deformation resistance is provided, such that a comparatively low flexion resistance occurs during plantar flexion of the foot part 10.

The distances between the spring elements 31, 32, 33 can be chosen such that all the spring elements 31, 32, 33 come to bear on one another simultaneously, and therefore an abrupt increase in the resistance to dorsal flexion is provided. If the spring elements 31, 32, 33 come into contact one after another on account of a corresponding spacing, there is a gradual increase in the resistance to dorsal flexion by the spring 30.

Between the spring elements 31, 32, 33, spacers can be arranged which are substantially rigid or alternatively deformable, in particular elastic. These spacers or spacer elements can be exchangeable and movable or can be arranged at different positions on the respective spring elements 31, 32, 33. By way of the spacers, it is possible to define and change the time and place of contact and force transmission between two spring elements 31, 32, 33. The nature of the force transmission can also be changed. If rigid spacers or spacer elements are used, direct force transmission takes place when contact with the spring elements is present. If the spacers or spacer elements are deformable, some of the force to be transmitted is applied for the deformation of the spacers or spacer elements, so that damping and, if necessary, energy storage takes place in the spacers. Thus, the increase in the deformation resistance can occur gently and less abruptly by coupling several spring elements to one another.

A variant of the spring is shown in FIG. 3, in which a plurality of spacers 40 are arranged spaced apart from one another on the base spring element 31, with clearances along the longitudinal extent of the base spring element 31. The spacers 40 establish a minimum distance of the base spring element 31 from a force transmission element 60 which, in the exemplary embodiment shown, is designed as a flexible, tensionally rigid and pressure-yielding tension means, for example a belt, cable or rope. Alternatively, the force transmission element 60 can also have an elasticity. If the upper end of the spring element 31 is bent to the left, while a lower end is fixed, this results in the spring element 31 curving. The spacers 40 arranged fixedly on the spring element 31 fan out, and the force transmission element 60 is placed under tension, since the path between the two ends of the force transmission element 60 increases on account of the bending. This leads either to an elastic extension of the force transmission element 60 or to a compression of the spacers 40 and, in both cases, to an increase in the resistance against further bending of the spring 30. If the upper end deforms and bends to the right, the right-hand ends of the spacers 40 move toward each other, the distance between spacers 40 decreases, and the force transmission element 60 is deformed on account of the flexibility. If a tensile stress is present within the force transmission element 60, it is reduced. For example, in an embodiment as a rope or belt, the latter folds up. The resistance to bending to the right in the direction of a plantar flexion of the foot part 10 is thus made available by the spring element 30 alone and is therefore lower than during an oppositely directed bending for a dorsal flexion of the foot part 10.

As an alternative to the design of the force transmission element 60 as a belt or rope, it can also be designed as a telescopic element. All the force transmission elements can be coupled to an actuator in order to achieve a change of the effective length.

FIG. 4 is a detail view of a section of a spring 30 with a plurality of spring elements 31, 32, 33. Clearances are formed between the spring elements 31, 32, 33, such that the spring elements 31, 32, 33 do not touch in the neutral position shown. The spring 30 is designed in one piece such that, above the clearances between the spring elements 31, 32, 33, a continuous material region is present, in particular made of a fiber composite material. The spring 30 has a slight curvature, so that, as explained with reference to FIG. 2, bending to the left results in an increased resistance and an associated greater moment against a deformation of the spring 30, whereas, upon bending to the right, a lower moment is needed to bring about a deformation. In the exemplary embodiment shown, the radii of curvature of all the spring elements 31, 32, 33 are equal in size; in one variant the radii of curvature are different. Likewise, the distances between the spring elements 31, 32, 33 can be non-uniform and can also change over the length of the spring elements 31, 32, 33.

FIG. 5 is a schematic sectional representation of a spring 30, in which a force transmission element in the form of a tensioning means 40 is secured to the spring 30 at two mutually spaced apart ends. A spacer 60 is designed in an undulating shape and is arranged between the spring 30 and the tensioning means 40. The spacer 60 has no or very little bending stiffness, so that very stable maintenance of a distance between the force transmission element 40 and the spring 30 is achieved without the bending properties and deformation resistances of the spring 30 being affected by the spacer 60. The material of the spacer 60 is stable to pressure in the exemplary embodiment shown, so that, on account of the almost vertically oriented portions between the spring 30 and the spacer 40, the force transmission element 40 is largely prevented from approaching in the direction of the spring 30. If the spring 30 is bent upward with its two ends, a bending resistance results solely from the bending stiffness of the spring 30. In an oppositely directed movement, the tensile means 40 is tensioned and causes an increase in the bending stiffness of the spring 30, the degree of the increase in the bending stiffness being dictated by elastic properties of the force transmission element 40 and, optionally, the spacer 60. The higher the elasticity of the force transmission element 40, the lower the increase in the bending stiffness of the spring 30; the same applies to the elasticity of the spacer 60.

FIG. 6 shows a variant of the spring 30 according to FIG. 5. In the exemplary embodiment, only the right-hand end of the force transmission element 40 is fixed to the right-hand end of the spring 30, whilst the left-hand end is coupled to an actuator 50. The actuator 50 can be designed, for example, as a motor with a gearing and a spindle for winding up the force transmission element 40. Alternative actuators such as linear actuators, magnetic switches, levers, mushroom elements or the like can be arranged on the spring 30 or in another component of the orthosis and connected to the force transmission element 40. The tension or the effective length of the force transmission element 40 can be changed via the actuator 50. If the effective length is shortened and the force transmission element 40 is pulled to the left, for example by being wound up, the tension within the force transmission element 40 increases and thus also the bending stiffness of the entire construction against downward bending; upward bending of the two ends of the spring 30 is thereby facilitated.

Alternatively or in addition, an actuator 50 is assigned to the spacer 60 in order to move the spacer 60 or change its properties. In the exemplary embodiment shown, the spacer 60 is moved away from the spring 30 in some areas, as a result of which the distance between the two opposite ends of the force transmission element 40 increases and a corresponding increase in tension and an increase in overall stiffness is achieved. If the spacer 60 is pulled in the direction of the spring 30, the distance between the two end points of the force transmission element 40 decreases and a tension that is optionally present within the force transmission element 40 is reduced. Both by changing the effective length or position of the force transmission element 40 and by changing the position or the elastic properties or deformation properties of the spacer 60, it is possible to adjust the bending stiffness of the spring 30. The spacer 60 can be filled, for example, with a magnetorheological fluid which, by application of a magnetic field, can be changed in terms of viscosity and thus compliance, such that the bending stiffness of the spring 30 is thereby changed. The coil for generating or changing the magnetic field is then the actuator. Depending on the situation, the adjustment can be performed by the user of the orthosis, by the orthopedic technician or even by artificial intelligence. Alternatively or in addition, sensors are assigned to a control device and transmit sensor values to the control device. The actuators 50 are activated or deactivated on the basis of the transmitted sensor values. The sensors are arranged on the orthosis, at least on the user, and transmit data which in particular represent the load, the gait situation and/or the environment.

1. An orthosis, comprising:
a foot part, and
a lower-leg part, and
a spring, wherein the spring connects the foot part and the lower-leg part, and wherein, starting from a neutral position, the spring provides a higher resistance during dorsal flexion than during plantar flexion.

2. The orthosis as claimed in claim 1, further comprising a force transmission element, wherein the spring is coupled to the force transmission element and the force transmission element transmits forces only when the foot part is moved in a direction of dorsal flexion.

3. The orthosis as claimed in claim 2, wherein the force transmission element is designed as a tension rod, cable, belt or telescopic rod.

4. The orthosis as claimed in claim 2, wherein the force transmission element is designed for mounting or is mounted in an adjustable manner.

5. The orthosis as claimed in claim 2, further comprising one or more actuators, wherein the force transmission element is assigned to an actuator of the one or more actuators, wherein a location of force transmission provided by the force transmission element and/or a time of the force transmission provided by the force transmission element is changeable by the actuator.

6. The orthosis as claimed in claim 1 wherein the spring comprises has a plurality of spring elements.

7. The orthosis as claimed in claim 6, wherein each of the plurality of spring elements are arranged spaced apart from one another in a neutral position and bear against one another during dorsal flexion.

8. The orthosis as claimed in claim 6 wherein the plurality of spring elements bear on one another in succession during dorsal flexion.

9. The orthosis as claimed in claim 6 further comprising one or more spacers arranged between each of the spring elements in the plurality of spring elements.

10. The orthosis as claimed in claim 9, wherein at least one spacer of the one or more spacers is mounted movably on at least one of the plurality of spring elements.

11. The orthosis as claimed in claim 8, further comprising one or more actuators, and wherein at least one spacer of the one or more spacers is assigned to an actuator of the one or more actuators.

12. The orthosis as claimed in claim 6 wherein at least one of the plurality of spring elements is are designed as a leaf spring.

13. The orthosis as claimed in claim 6 wherein the plurality of spring elements are arranged parallel to one another and, wherein in a neutral position of the orthosis, the plurality of spring elements are shaped in an arch curved counter to a direction of walking.

14. The orthosis as claimed in claim 6 wherein at least two of the plurality of spring elements have different spring stiffnesses.

15. The orthosis as claimed in claim 1 wherein the spring comprises a base spring element on which the foot part and the lower-leg part are arranged.

16. The orthosis as claimed in claim 15, further comprising at least one further spring element mounted exchangeably on the base spring element.
